(19) United States
(12) Patent Application Publication
(10) Pub. No.: US 2021/0125725 A1
(43) Pub. Date: Apr. 29, 2021

(54) Automated Medical Device Regulation Risk Assessments

(71) Applicant: International Business Machines Corporation, Armonk, NY (US)

(72) Inventors: Corville O. Allen, Morrisville, NC (US); Isaac Erickson, Mansfield, TX (US); Craig A. Bennett, Woodstock, GA (US); Maren Erickson, Mansfield, TX (US); Justin L. Sheppard, Holly Springs, NC (US); Mark A. Laney, Wake Forest, NC (US)

(21) Appl. No.: 16/667,358

(22) Filed: Oct. 29, 2019

(51) Int. Cl. G16H 50/30; G16H 15/00; G16H 50/70; G16H 70/20; G16H 10/60

(52) U.S. Cl. CPC G16H 50/30; G16H 15/00; G16H 10/60; G16H 70/20; G16H 50/70

(57) ABSTRACT

Mechanisms are provided for automated medical device regulation risk analysis (MDRRA). An automated MDRRA engine receives compliance rules for a medical device, a data dictionary for a patient data source, and medical device characteristics data for the medical device. The automated MDRRA engine automatically determines, based on the compliance rules and the data dictionary, critical fields of patient data and medical device characteristics data for demonstrating medical device efficacy according to the compliance rules. The automated MDRRA engine automatically determines, based on the critical fields, one or more patient population data subsets for a plurality of patients from a patient population data set and selects a population segment from the one or more patient population data sets to populate an electronic case report form (eCRF) study build. The automated MDRRA engine automatically populates fields of the eCRF with data from the population data sets corresponding to the selected population segment.

### BACKGROUND

The present application relates generally to an improved data processing apparatus and method and more specifically to mechanisms for automated medical device regulation risk assessment.

Most governments, and many industry organizations, professional organizations, and the like (referred to collectively herein as “oversight organizations”), have promulgated regulations for specifying required configurations, operations, and uses of medical devices so as to promote the efficacy of the medical procedures and treatments employing these medical devices, as well as the safety and welfare of patients being treated. For example, the United States Food and Drug Administration (FDA) is the oldest comprehensive consumer protection agency in the United States and has responsibility for regulating medical devices, such as under various regulations as the Medical Device Amendments to the Food, Drug, and Cosmetic Act in 1976, the Safe Medical Devices Act (SMDA) of 1990, Mammography Quality Standards Act (MQSA) of 1992, the Food and Drug Administration Modernization Act (FDAMA) of 1997, the Medical Device User Fee and Modernization Act (MDUFMA) of 2002, and most recently in the 2017 Food and Drug Administration Reauthorization Act (FDARA).

Complying with such regulations is an expensive and resource intensive process. These costs are amplified when new regulations are passed that require existing medical devices to be recertified under the new regulations. Complying with medical device regulations is typically done as a pre-market step, such as by performing a clinical trial. However, many new and updated regulations are effective with regard to all medical devices, both new and medical devices already in the market.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method, in a data processing system comprising a processor and a memory, the memory comprising instructions which are executed by the processor to cause the processor to implement an automated medical device regulation risk analysis (MDRRA) engine. The method comprises receiving, by the automated MDRRA engine, compliance rules for a medical device, wherein the compliance rules specify parameters for medical device efficacy, and receiving, by the automated MDRRA engine, at least one data dictionary for a patient data source and medical device characteristics data for the medical device. The method further comprises automatically determining, by a field orchestration engine of the automated MDRRA engine, based on the compliance rules and the at least one data dictionary, critical fields of patient data and medical device characteristics data for demonstrating medical device efficacy in accordance with the compliance rules. Moreover, the method comprises automatically determining, by a rules and fields analysis engine of the automated MDRRA engine, based on the critical fields, one or more patient population data subsets for a plurality of patients from a patient population data set. In addition, the method comprises automatically selecting, by the rules and fields analysis engine of the automated MDRRA engine, a population segment from the one or more patient population data sets to populate the electronic case report form (eCRF) study build. Furthermore, the method comprises automatically populating, by an eCRF generation engine of the automated MDRRA engine, fields of the eCRF with data from the population data sets corresponding to the selected population segment.

In some illustrative embodiments, receiving the at least one data dictionary further comprises mapping, by the field orchestrator engine of the automated MDRRA engine, a data dictionary for a patient data source, and medical device characteristics of a medical device, to common schema fields of a common schema. In some illustrative embodiments, automatically determining critical fields of patient data and medical device characteristics data for demonstrating medical device efficacy in accordance with the compliance rules further comprises analyzing, by the field orchestrator engine, the compliance rules and the common schema fields for congruence to produce rules metric data and critical fields data, wherein the rules metric data specifies checks against one or more thresholds for identifying normal/abnormal values in patient data, and wherein the critical fields data specifies critical fields in the patient data and medical device characteristics for evaluating the rules metric data.

In some illustrative embodiments, automatically selecting a population segment further comprises analyzing, by the rules and fields analysis engine of the automated MDRRA engine, the one or more patient population data subsets at least by matching portions of the patient population data set to one or more of the compliance rules to identify adverse event specific cases and population counts, and determining, by the rules and fields analysis engine, based on the identified adverse event specific cases and population counts, a statistically significant population segment required for specific adverse event cases based on the rules metric data for the critical fields.

In some illustrative embodiments, generating the eCRF further comprises selecting, by the rules and fields analysis engine, a subset of the critical fields and corresponding patient data fields in the patient data for an original medical device study, and generating, by the eCRF generation engine, the eCRF based on the subset of critical fields and corresponding patient data fields.

In some illustrative embodiments, selecting the population segment comprises determining, for a specific adverse event type, the population segment based on at least one threshold for population segments per compliance rule and critical field. In some illustrative embodiments, selecting a population segment comprises selecting a population segment in which statistically significant field criteria is within a specified manufacturer risk tolerance threshold, based on at least one of sampling size, standard deviation, significance level.

In some illustrative embodiments, automatically determining, by the rules and fields analysis engine of the automated MDRRA engine, based on the critical fields, one or more patient population data subsets for a plurality of patients from a patient population data set comprises matching one or more of the critical fields to portions of patient population data in the patient population data set, and performing a statistical analysis of the patient population data, in the patient population data set, to determine one or more patient population data subsets meeting one or more criteria specifying statistically significant subsets. In some illustrative embodiments, automatically selecting, by the rules and fields analysis engine of the automated MDRRA engine, a population segment from the one or more patient population data sets to populate the eCRF study build comprises generating a union of the one or more patient population data subsets. In some illustrative embodiments, the matching is performed for portions of the patient population data set corresponding to a specified injury type.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example block diagram of the primary operational elements of an automated medical device reporting (medical device) engine and corresponding data flow in accordance with one illustrative embodiment;

FIG. 2 is an example diagram of a portion of a data dictionary in accordance with one illustrative embodiment;

FIG. 3 is an example diagram of a medical device characteristics data structure in accordance with one illustrative embodiment;

FIG. 4A is an example diagram of a medical device rules data structure in accordance with one illustrative embodiment;

FIG. 4B is an example diagram of a rule metrics data structure in accordance with one illustrative embodiment;

FIG. 4C illustrates example data dictionaries for different patient EMR sources;

FIG. 4D shows an example of a corresponding portion of a common schema for the attribute fields of the data dictionaries represented in FIG. 4C, in accordance with one illustrative embodiment;

FIG. 4E provides examples of data records from source A and source B, respectively, in accordance with one illustrative example and one illustrative embodiment;

FIG. 4F provides an example diagram illustrating the combination and normalization of the data records shown in FIG. 4E in accordance with a common schema, and in accordance with one illustrative embodiment;

FIG. 5A is an example diagram of a portion of a population data sets database in accordance with one illustrative embodiment;

FIG. 5B is an example diagram of a manufacturer risk tolerance profile in accordance with one illustrative embodiment;

FIG. 6A-6C are example diagrams of portions of an automatically generated electronic case report form (eCRF) in accordance with one illustrative embodiment;

FIG. 7 is a flowchart outlining an example operation of an automated medical device reporting (medical device) engine in accordance with one illustrative embodiment;

FIG. 8 is an example diagram of a distributed data processing system in which aspects of the illustrative embodiments may be implemented; and

FIG. 9 is an example block diagram of a computing device in which aspects of the illustrative embodiments may be implemented.

### DETAILED DESCRIPTION

As mentioned above, various oversight organizations promulgate regulations for governing manufacturers and providers of medical devices so as to promote the general welfare and safety of patients. The United States Food and Drug Administration (FDA) is one such oversight organization, however, other governments and organizations, such as the European Union, have their own regulations regarding medical devices with which medical device manufacturers and providers must comply. Oversight organizations often modify these requirements. For example, effective May 25, 2020, medical device manufacturers must meet new European Union regulations.

As such requirements are changed, medical device manufacturers are required to expend additional monetary and human resources to be aware of and comply with these ever-changing regulations. In order to comply with such new requirements, for example, medical device manufacturers either directly load data sets into a static database, resulting in audit findings (i.e. oversight organizations will audit the manufacturers) and not meeting the intent of the new regulations, or run additional studies of the medical device in order to generate additional data sets that provide the necessary data for complying with the new regulations, which adds substantial costs in complying with the new regulations. Both solutions have significant risks and costs by being dependent on manual processes that could cost manufacturers millions of dollars in order to be able to bring a medical device to market. The risks may include failure to meet audit standards, fines, additional time spent documenting corrective actions, further review, or prevention of the medical device entering the corresponding market. These costs may reach tens of millions of dollars per year per device manufacturer and may be effectively hundreds of millions of dollars over a 10 year period. Complying with new and evolving medical device regulations will prove more and more costly as time goes on, with these current “solutions” becoming cost prohibitive and failing to be effective.

The illustrative embodiments provide an improved data processing apparatus and method that implements automated data processing mechanisms for automatic risk assessment for medical devices, where the risks that are assessed are the risks that the medical device will not comply with medical device regulations. The mechanisms of the illustrative embodiments greatly reduce any manual efforts for complying with medical device regulations, especially new regulations. The improved data processing system mechanisms of the illustrative embodiments implement automated improved synthetic data transformations based on one or more data dictionaries for patient data sources and medical device characteristic information analysis, as well as regulatory rules definitions, to deduce the critical fields in already stored patient population data that may be used to drive population of electronic case report forms (eCRFs) and thereby generate a study build for complying with medical device regulations. The eCRF is an electronic form for capturing data in medical device clinical trials. For a single clinical trial, a collection of eCRFs is created using an electronic data capture (EDC) system. Patient information is collected during the clinical trial and used to populate the eCRF, where this patient information includes, but is not limited to, adverse events. The eCRFs are customized to meet the needs of the particular clinical trial. The data capture in the eCRFs is owned by the sponsor and is reviewed, analyzed, and summarized prior to submission to the oversight organization.

The study build is the development process that creates a collection of eCRFs, organized within a defined timeline of procedures. A study build is all of the data capture needs for a specific clinical trial. The study build, in accordance with the mechanisms of the illustrative embodiments, is a repository in which mined data from patient population data sets is housed which allows the sponsor to easily review, analyze and summarize the data.

The illustrative embodiments automatically generate the eCRFs based on one or more selected subsets of patient population data to thereby simulate new medical device studies or clinical trials tied to the medical device, while employing regulatory statistically significant thresholds for proving regulatory compliance based on a manufacturer risk tolerance profile. The manufacturer risk tolerance profile is created in conjunction with the medical device manufacturer and is based on a number of factors including the medical device's risk classification under the oversight organizations' regulations (e.g., FDA risk classifications), manufacturer's view of the safety of a device, and the manufacturer's weighting of the cost of manual review versus the risk of misrepresenting the total population of patient data. The manufacturer's risk tolerance profile is used in conjunction with the rules and fields analysis engine, described hereafter, to produce a sample data set for manual review.

The mechanisms of the illustrative embodiments may be implemented when new medical device regulations or modified medical device regulations are promulgated. These new and/or modified medical device regulations may be converted to rules data structures which are then used to identify critical fields for eCRF generation. These critical fields may then be used to extract case data from already collected and stored patient population data, using a statistically significant sub-portion of the patient population data, to generate new case data for populating the eCRFs. The mechanisms of the illustrative embodiments thus, provide automated mechanisms simulating case studies for complying with new and/or updated medical device requirements as they are promulgated. This greatly reduces the resource costs for medical device manufacturers to comply with evolving regulations of oversight organizations.

It should be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

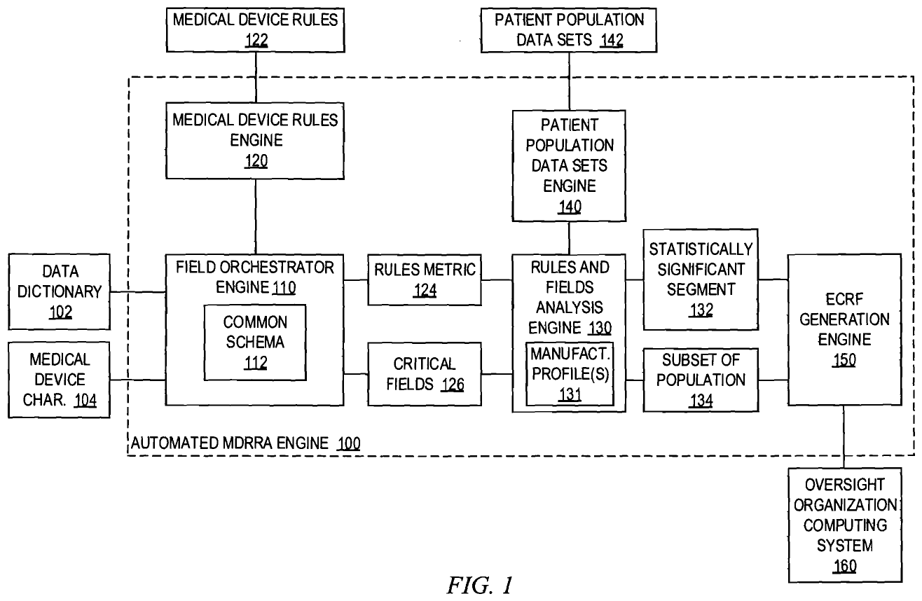

As noted above, the illustrative embodiments provide an improved data processing apparatus and method that implements automated data processing mechanisms for automatic medical device regulation risk assessment with electronic case report form (eCRF) and study build generation that greatly reduces any manual efforts for complying with medical device regulations, especially new regulations. FIG. 1 is an example block diagram of the primary operational elements of an automated medical device regulation risk assessment (MDRRA) engine and corresponding data flow in accordance with one illustrative embodiment. As shown in FIG. 1, the automated MDRRA engine 100 comprises a field orchestrator engine 110, a medical device rules engine 120, a rules and fields analysis engine 130, a patient population data sets engine 140, and an eCRF generation engine 150.

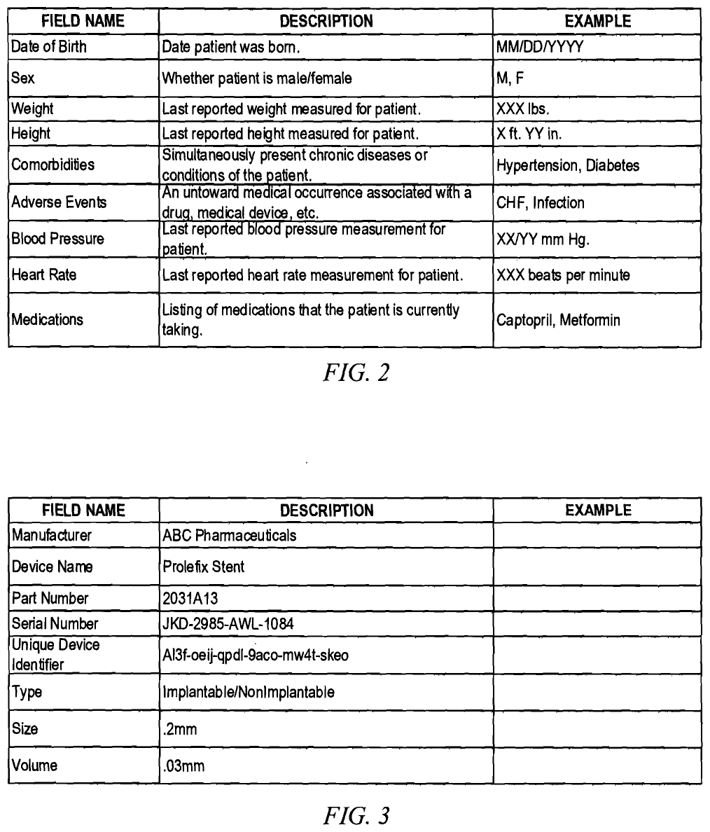

The field orchestrator engine 110 comprises computer logic that operates to identify and map data dictionary 102 data fields to common schema data fields of a predefined common schema 112 with which the field orchestrator engine 110 is configured, and based on matching fields of rules as identified by the medical device rules engine 120. The data dictionary 102 specifies the metadata of patient population data structures for patient cases, e.g., metadata describing particular fields provided in patient electronic medical records (EMRs), from a particular patient data source. It should be appreciated that there may be different data dictionaries 102 for different sources of patient data and thus, different mappings of such data dictionaries 102 to corresponding common schema fields, e.g., mapping a field of “gender” in the data dictionary 102 to a common schema field of “sex.” FIG. 2 is an example diagram illustrating one example of a portion of a data dictionary 102 in accordance with one illustrative embodiment. As shown in FIG. 2, this data dictionary 102 comprises various personal data about the patient including their date of birth, sex or gender, etc., as well as clinical data including the patient's weight, height, blood pressure, heart rate, comorbidities associated with the patient, any adverse events reported, medications the patient is taking, etc. The data dictionary 102 gives a full description of each field, any relevant formatting characteristics, and for coded fields, the controlled vocabulary and codings, e.g., 1=female, 2=male, 3=Other, 4=not specified.

The definition of the fields present in the patient data, e.g., patient EMR data, for a particular source, as specified in the data dictionary 102, may then be used to map the patient data to corresponding fields of a common schema representation of the patient data, e.g., mapping patient data in a “sex” field in the source EMR data to a corresponding “gender” field in a common schema representation of the EMR data. Similarly, a format of the height data for a patient present in a “height” field of the EMR data may be mapped to a similar “height” field of the common schema, however the format of the data may be changed, e.g., changing from a feet, inches format to simply an inches format, e.g., 5 feet, 10 inches would be converted to 70 inches, or even to another unit of measure such as centimeters, for example, e.g., converting from English measurements to metric measurements. Essentially, the field orchestrator engine 110 provides logic for converting data to a common representation corresponding to the common schema so that the data is compatible regardless of the particular fields and formats used by the source computing systems from which the data is received.

The field orchestrator engine 110 further comprises computer logic that operates to identify and map medical device characteristics 104 data fields to common schema 112 data fields. The medical device characteristics 104 specifies the characteristic data for a medical device, e.g., unique medical device identifier, medical device name, manufacturer information, and other characteristics data that may be used to identify the medical device in medical insurance claims data, patient electronic medical record (EMR) data, or other sources of patient population data. FIG. 3 is an example diagram of a medical device characteristics data structure in accordance with one illustrative embodiment. As shown in FIG. 3, the medical device characteristics data structure 104 comprises manufacturer information 310 and medical device specific characteristic information 320, such as the device name, part number, serial number, unique device identifier, type of device, size, volume, etc. In one illustrative example, the manufacturer information 310 comprises the name, street, locality, postcode, country, and contact information for the manufacturer. As a further example, the medical device specific characteristic information 320 may also comprises a nomenclature code, device name/make, risk category, reusable device indicator (yes/no), animal or human tissue indicator (yes/no), UDI, certificate, certificate number, certificate type, data of issue, expiration date, manufacturer and authorized representative, notified body, general scope description and details on device, etc.

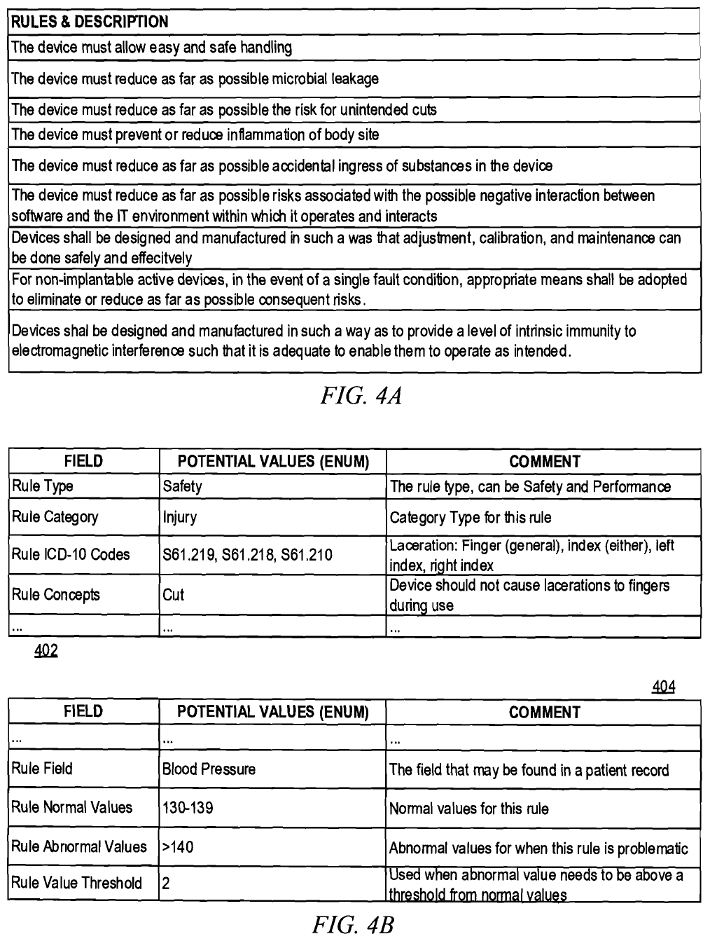

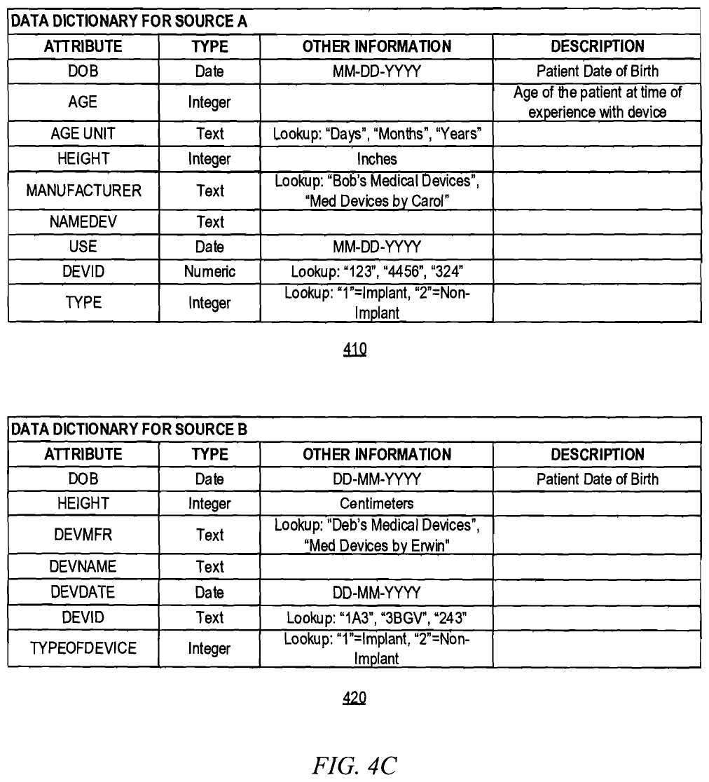

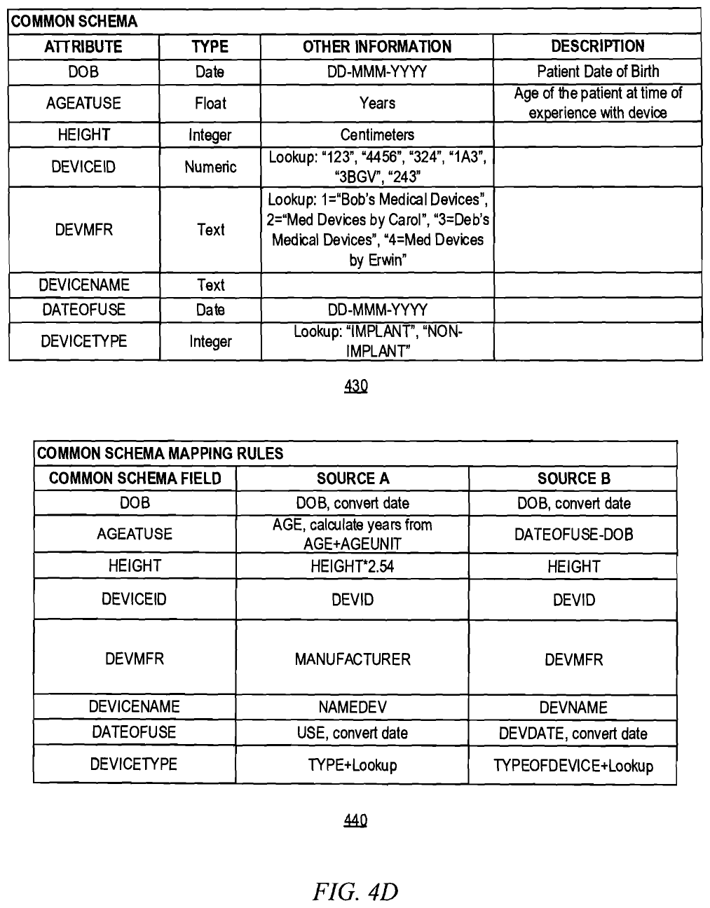

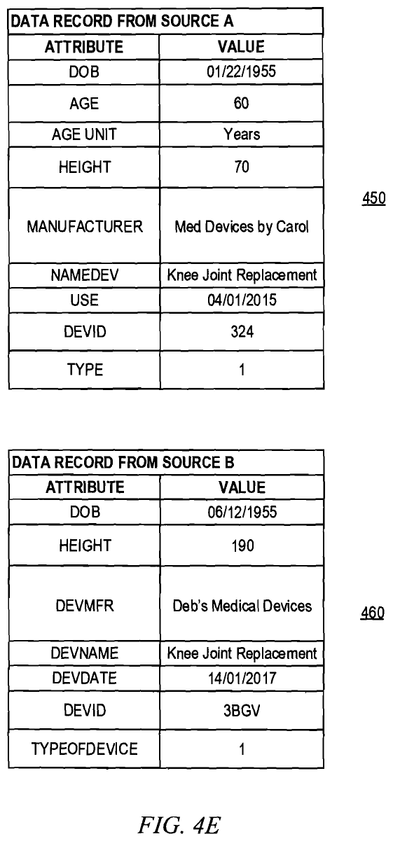

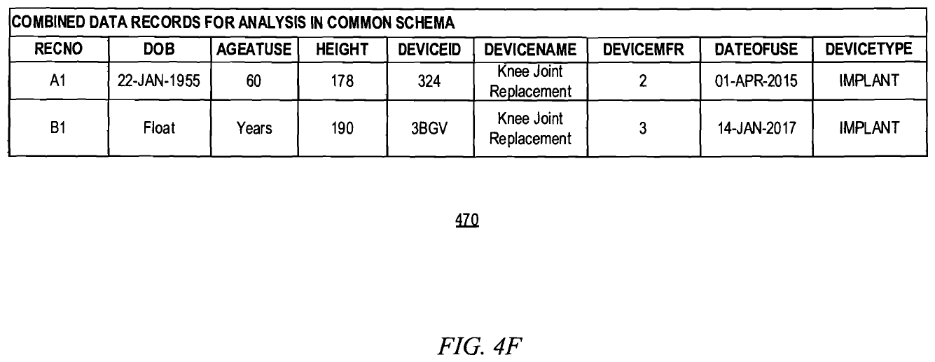

The field orchestrator engine 110 also comprises computer logic that operates to interface with the medical device rules engine 120 to compare features of medical device rules data structures 122 with the mapped fields of the data dictionary 102 and medical device characteristics 104 in the common schema 112. The medical device rules data structures 122 are data structures specifying the required patient and medical device characteristic fields for medical device compliance, as well as relationships between these required fields. The medical device rules data structures 122 may be converted to one or more rules metrics 124 by the medical device rules engine 120 and provided to the field orchestrator engine 110 for use in matching fields of the data dictionary 102 and medical device characteristics 104 to fields of the corresponding rules data structures 122. FIG. 4A is an example diagram representing example medical device rules data structures 122. FIG. 4B is an example diagram of a rules metric data structure 124 in accordance with one illustrative embodiment. In the depicted example, the rule metric data structure 402 of FIG. 4B is an example of a pulse meter, intended to be placed on an index finger, with the rule metric data structure corresponding to the rule “the device must reduce as far as possible the risk for unintended cuts” in FIG. 4A. The rule metric data structure 404 shows examples of additional fields that may be part of a rule metric data structure in accordance with illustrative embodiments for a particular type of medical device.

The medical device rules data structures 122 codify oversight organization rules for performing medical device reporting into computer system usable rules data structures. These medical device rules data structures 122 may be provided as pseudo conditional computer rule statements, may be provided as a specific rule language formatted rule data structures, or as natural language content defining the rule criteria for indication of a properly configured and performing medical device. The field orchestrator engine 110 analyzes the common schema fields to which the data dictionary 102 and device characteristics 104 have been mapped, and the rules specified in the medical device rules data structures for congruence and produces a rules metric data structure 124 and a critical fields data structure 126.

As noted above, the medical device rules data structures 122 may be provided as pseudo conditional computer rule statements or as specific rule language formatted rule data structures, or as natural language content. In one illustrative embodiment, for medical device rules data structures 122 provided as pseudo conditional computer rule statements or specific rule language formatted rule data structures, the field orchestrator engine 110 matches the medical device rule data structure 122 for injury, or adverse event, and maps to appropriate data categories in the common schema 112.

In one illustrative embodiment, for medical device rule data structures 122 provided as natural language content, natural language processing may be performed on the medical device rule data structure 122, by the medical device rules engine 120, to identify and extract the concepts and values specified in the natural language content and map those concepts and values to corresponding data categories in the common schema. In some cases, this mapping may be done by matching terms/phrases extracted from the medical device rule data structures 122 with terms/phrases present in descriptions of fields specified in the common schema 112, for example.

Thus, in some illustrative embodiments, the medical device rules 122 are processed by the medical device rules engine 120 to extract key terms/phrases and concepts and produce a set of rules metrics data structures 124 comprising these key terms/phrases and concepts, corresponding values or ranges of values, etc. all mapped to the common schema 112. These rules metrics data structures 124 may be generated by the medical device rules engine 120 prior to operation on a specific input data dictionary 102 and medical device characteristics dictionary 104 or may be retrieved from a database. Moreover, once generated, such rules metrics data structures 124 may be stored in such a database for later retrieval and use by the medical device rules engine 120 for new inputs of data dictionaries 102 and/or medical device characteristics 104.

The particular rules metrics data structures 124 utilized by the field orchestrator engine 110 may be selected by the medical device rules engine 120 based on the data dictionary 102 and medical device characteristics 104 which serve as input to the field orchestrator engine 110 so that the values can be normalized to the common schema 112. The field orchestrator engine 110 analyzes the data dictionary 102 and matches the fields of the data dictionary 102 to the medical device rules 122 based on a data dictionary fields-to-rule field or rule concept matching and then a set of rule metrics data structures 124 are selected based on this matching. Based on the selected rules metrics data structures 124, a set of critical fields 126 are selected that match the rules fields and align with the common schema 112. Thus, the critical fields 126 are the fields that are present in the common schema 112 and are specified in the medical device rules 122. This alignment of the rules metrics data structures 124 contains normal/abnormal values, what type of rule the normal/abnormal values are in, the threshold(s) used, etc. and these are included in the critical fields 126.

As will be described in greater detail hereafter, the critical fields 126 and the rules metrics data structure 124 values are used to form queries, including base patient data (e.g., patient age and ethnicity), to query the patient population data sets 142. That is, as described hereafter, the rules metrics data structures 124 and critical fields 126 are used by the rules and fields analysis engine 130, using the patient population data sets 142 to produce one or more statistically significant segments of patient population data based on statistical analysis. For example, a null hypothesis analysis, based on a critical field for blood pressure, may be performed to determine whether a portion of patient population data is within the range expected for a sample size or not. The results of such analysis are processed by the rules and fields analysis engine 130 to produce statistically significant segments 132 based on the query for fields that are determined to be of importance to representing medical device compliance with regulations, i.e. critical fields, as represented by the rules metrics data structure 124 and critical fields 126. Manufacturer profiles 131 may be utilized by the rules and fields analysis engine 130 to represent the manufacturer's risk tolerance level indicating how much risk the medical device manufacturer is willing to take with regard to complying with regulations, i.e. how much risk the manufacturer is willing to take that existing patient population data sets 142 will satisfy regulations. The statistically significant segment(s) 132 and a subset 134 of the patient population data sets 142 are input to an ECRF generation engine 150 and, either combined or individually, are used by the ECRF generation engine 150 to take values from the fields of the subset of patient population data sets 134 and use a data import operation to populate one or more ECRFs with the values to create a study build. The study build is used in clinical development by an oversight organization computing system 160, where a review or a simulation or a study can be performed to demonstrate compliance of the medical device with the regulations represented.

Returning to the operation of the field orchestrator engine 110, to illustrate the mapping to the common schema 112 performed by the field orchestrator engine 110 further, consider the examples of data dictionaries 102 for two sources of patient EMR data, and the transformation and normalization of the data with regard to a common schema 112 as shown in FIGS. 4C-4F. FIG. 4C illustrates example data dictionaries for different patient EMR sources, referred to as sources A and B, respectively. The data dictionary 410 for source A has particular attributes provided in the patient EMR which different from the attributes in the data dictionary 420 for source B, e.g., MANUFACTURER in data dictionary 410 corresponds to DEVMFR in data dictionary 420. Moreover, in some cases, the types and formats (indicated in “other information” in the data dictionaries 410 and 420), may differ between the data dictionaries 410 and 420 as well.

FIG. 4D shows an example of a corresponding portion of a common schema 430 for the attribute fields of the data dictionaries 410, 420 represented in FIG. 4C. FIG. 4D also shows common schema mapping rules 440 for mapping the attributes in the data dictionaries 410 and 420 to the common schema field indicated in the common schema 430. The attributes in the common schema 430 may differ from those in either of the data dictionaries 410, 420, e.g., rather than using NAMEDEV or DEVNAME from the data dictionaries 410, 420, the common schema uses the attribute DEVICENAME. The common schema mapping rules 440 provides the rules for mapping the attributes from the source data dictionaries 410, 420 to the common schema attributes, e.g., AGEATUSE in the common schema 430 is obtained from data dictionary 410 by calculating years from AGE+AGEUNIT and is obtained from the data dictionary 420 by calculating DATEOFUSE-DOB. For some attributes, a format conversion function is employed to convert formats from one format to another, e.g., converting dates from one date format to another.

FIG. 4E provides examples of data records 450, 460 from source A and source B, respectively. Data records 450, 460 have the same attributes as specified in their respective data dictionaries 410, 420 but have specific values for demonstrating the combination and normalization of these data records as shown in FIG. 4F. As shown in FIG. 4F, values for the combined records are normalized and, in the case of AGEATUSE, the value is calculated from DOB and DEVDATE from the data record from source B 460 for record number (RECNO) B1.

As shown in the example of FIGS. 4C-4F, some fields in the common schema 112 may be derived from two (2) or more fields in a data source corresponding to a data dictionary 102. For example, Source B does not contain a field that maps to “AGEATUSE” and the age of the patient at the time they encounter the device may have significant impact on the safety and/or efficacy of the device. Because this is an important field, it is important to have the corresponding value in the combined and normalized records in the common schema. Fortunately, the Patient's date of birth (“DOB”) and the date at which the patient encountered the device (“DEVDATE”) are provided. Using these two fields, the field orchestrator engine 110 is able to calculate “AGEATUSE” from the source data and populate the common schema.

As noted above, the field orchestrator engine 110 generates a rules metric data structure 124 and critical fields 126 based on the mapping of the data dictionary 102 and medical device characteristics 104 to the common schema 112 and identifying which fields are critical to demonstrating medical device compliance with regulations, as determined by matching fields of the data dictionary 102 and medical device characteristics 104 to fields extracted from medical device rules 122 by the medical device rules engine 120. The rules metric data structure 124, an example of which is again provided in FIG. 4B, provides content specifying one or more checks of patient and medical device information against acceptable thresholds for normal/abnormal values of particular data types. For example, the rules metric data structure 124 may specify, for one or more readings generated by the medical device, a normal range for the readings as well as an abnormal range for these readings. Moreover, the rules metric data structure 124 may also specify various medical device rules data structure 122 concepts specified in the medical device rules data structure 122 that are associated with normal or abnormal operation of the medical device or regarding injuries associated with the medical device, e.g., laceration.

The critical fields data structure 126 specifies the fields in the common schema 112 that are critical to demonstrating compliant medical device performance in accordance with the medical device rules data structures 122. These critical fields are fields of the data dictionary 102 and the medical device characteristics 104 that map to common schema 112 fields and which are referenced by medical device rules data structures 122. That is, for a particular device, specific fields may be deemed critical. For example, the age of the patient may be very important for a stent, as the usefulness of that device in a child will have a much shorter time span as the child grows than it would in an older patient. In terms of the rule metric data structure 124, this is a weighting on a particular field of a rule in the medical device rules 122. For example, the age of a patient with a pacemaker is always important, but if the patient has a comorbidity of CHF prior to the implant, that would be more important than the age in determining the cause of an adverse event (AE). Thus, the critical fields data structure 126 specifies which fields are critical to demonstrating proper performance of the medical device in accordance with the medical device rules data structures 122, while the rules metric data structure 124 specifies the normal and abnormal values, or ranges of values, of these various critical fields, or combinations of these critical fields.

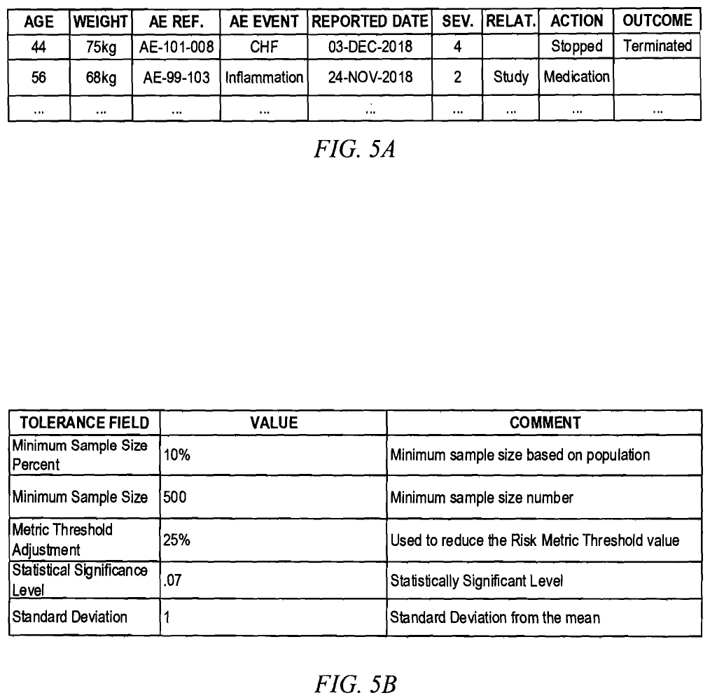

The rules metric data structure 124 and critical fields data structure 126 are provided to the rules and fields analysis engine 130 which uses these data structures 124, 126 to analyze population data sets 140 accessed via the patient population data sets engine 140. The patient population data sets engine 140 obtains and maintains one or more population data sets 142 comprising patient data and/or incident data, such as medical insurance claims data specifying an injury or adverse event and associated patient data and/or medical device data. For example, the patient population data sets 140 may comprise medical insurance claims data that specifies a patient for which the medical insurance claim is made, identifiers, names or other identifying information for any medical devices involved in the medical insurance claim, any associated injuries or adverse events, and other medical insurance claims information. This information may be correlated with patient data, such as patient electronic medical records (EMRs), so as to obtain patient data for the patient specified in the medical insurance claims information. FIG. 5A is an example diagram of a portion of a population data set database in accordance with one illustrative embodiment.

The rules and fields analysis engine 130 uses the rules metric data structure 124 and critical fields data structure 126 to analyze the population data sets 142 by matching population data, e.g., medical insurance claims data and patient data, against critical fields and normal/abnormal values of critical fields for specific injury types specified in the rules metric data structure 124. That is, as part of the other concepts specified in medical device rules data structures 122 which are identified in the rules metric data structure 124, these concepts may include particular injury types associated with the medical device, which may be specified in any of a number of different ways including International Classification of Disease, tenth revision (ICD-10) codes, for example.

For example, for a specified injury type in the rules metric data structure 124 and/or the critical fields data structure 126, entries in the population data sets 142, e.g., medical insurance claims data, patient EMR data, site EMR data, etc., matching that specified injury type are identified by the rules and fields analysis engine 130. Population counts are generated for these matching entries and statistical processes are applied by the rules and fields analysis engine 130 for determine one or more subsets of the population data sets 142 that are statistically significant.

Thus, the field orchestrator engine 110 ingests patient data (data dictionary 102) and medical device data (medical device characteristics dictionary 104) and, by applying normalization and standardization techniques, combines the data into a common schema 112. This common schema 112 is used in an analysis process that is used to determine the entire set (population) of data records that indicate the targeted medical device was in use, the subset of data records that indicate that an adverse event (AE) or injury was associated with the medical device, and the category(ies) of adverse events or injuries by severity. Further, the analysis derives the predictive models by which data records are segmented for manual analysis as embodied by machine learning and/or rules-based algorithms. In addition, the mechanisms of the illustrative embodiments analyze the characteristics of the population dataset 142 and data subsets by device type, adverse event occurrence, and severity, to generate the statistically significant dataset for manual review, where the manufacturer risk tolerance profile is incorporated into the segmentation rules to adjust the meaning of “statistically significant” based on the nature of a failure of the medical device to induce adverse events or injuries and the manufacturer's position regarding the parametric evaluation of the sample set for manual review versus the subset of records reporting possible adverse events or injuries.

In one illustrative embodiment, statistical significance of data corresponding to a medical device is analyzed relative to a control of a baseline medical device of a similar device type. Thus, statistical values are generated for the various critical fields 126 across the population data sets 142 for the target medical device, i.e. the medical device for which medical device is being performed, as identified in the device characteristics data structure 104. The statistical values are compared to the baseline, or “control,” medical device's values for similar fields to determine the most critical and significant fields.

In one illustrative embodiment, the statistical significance of fields in the critical fields data structure 126 as determined based on the population data sets 142 may be determined using one or more of the following statistical processes. First a dynamic testing of a null hypothesis around injury or adverse event types tied to the rules metric data structure 124 is performed. The null hypothesis significance testing (NHST) is a method of statistical inference by which an experimental factor is tested against a hypothesis of no effect or no relationship based on a given observation. In the context of the present invention, the null hypothesis is that the distribution of adverse events or injuries, including the number, type and severity, will be the same as that seen in the clinical trials and on-going safety reporting. If the population data collected for analysis differs, then the statistical significance analysis will determine the extent to which it differs and in what ways, e.g., which occurrences of adverse events/injuries, types of adverse events/injuries and/or severities of adverse events/injuries differ from what is seen in the clinical trials and on-going safety reporting. Based on the congruence of the population data to the known data, a base sample size is determined. Machine learning and rules based algorithms will then segment the data to be manually reviewed. The sample size, and an effective size, of the population across the population data sets 142 is determined. The sample size may be smaller than the effective size of the population data sets 142 where the device manufacturer, as specified in their risk profile, an example of which is shown in FIG. 5B, indicates that they are willing to accept a sample size smaller than the effective size due to the burden of reviewing the effective size. In such cases, the manufacturer indicates that they are willing to accept the risk of random sampling within the effective population to produce the ECRF data. Additionally, there may be cases where there is insufficient data available to build an effective population data set and thus, a smaller sample size may be utilized.

Another statistical process that may be utilized is A/B testing for adverse events/injuries against the baseline or control medical device for a particular device type, or specific medical device. A/B testing (also known as bucket tests or split-run testing) is a randomized experiment with two variants, A and B. A/B testing includes application of statistical hypothesis testing or “two-sample hypothesis testing” as used in the field of statistics. A/B testing is a way to compare two versions of a single variable, typically by testing a subject's response to variant A against variant B, and determining which of the two variants is more effective. For example, assume that there are two populations A and B from patient population data set 142. Population A represents a population of a similar medical device type as a control medical device, and population B represents a population utilizing the medical device being analyzed. The rules metric data structure 124 contains a rule field, which matches a critical field 126 representing inflammation due to device implant or graft. The existence of this adverse event needs to be confirmed on whether it is statistically significant compared to the control population A against population B using NB testing.

Many other types of statistical analysis processes may be utilized to determine the statistical significance of fields in the critical fields data structure 126 as determined based on the population data sets 142, without departing from the spirit and scope of the present invention. For example, various standard deviation based statistical analyses may be applied for field values of interest to determine those that are statistically significant.

Thus, the critical fields 126 are the cross-section of fields that are found in a set of rules metric data structures 124 for a device type. The analysis engine 130 scans the rules metrics data structures 124 for fields that correspond to the critical fields 126 and selects a subset of fields to query the population data set 142 using the population data set engine 140. The population data set engine 140 performs a combination of AND and OR logic operations for one or more critical fields 126 to build a number of population data subsets 134. This can be performed by pulling data from a population data set 142, for example, population claims data set, building a query to represent a subset 134. For example, the fields of Age>50 and Inflammation of the Breast ICD-10 code T85.9 may be used to produce one population data subset 134 (it is assumed that the device type is included in the query and if necessary the manufacturer and unique device identifier (UDI) of the device). Hence, one or more subsets 134 of the population data sets 142 are selected based on the rules metric data structure 124 and the most critical and statistically significant fields. In one illustrative embodiment, a union of the population data subsets 134 across the most critical fields 126 and the statistically significant sets for the rules metric data structure 124 that are of most importance to the governing body is generated.

Using empirical data, the rules and fields analysis engine 130 establishes a threshold for the population segments per rule and critical fields over the system produced segment. This is used to better tune the system for certain fields to increase the segment for better success with governing bodies. After determining the one or more subsets of the population data sets 142 that are associated with the most critical and significant fields of the rules 122 for medical device based on the rules metric data structure 124 and critical fields data structures 126, a subset of critical fields that is important and the default base patient fields for a study are selected. Examples of base patient fields include, but are not limited to, age, gender, ethnicity and weight, i.e. typical general patient data fields.

Selection rules for selecting the subset of patient population data can be adjusted for a medical device manufacturer's tolerance for risk, balanced with the cost of performing a study. That is, manufacturers of medical devices may establish tolerance profiles for medical devices that specify the manufacturer's position regarding the parametric evaluation of the sample set for manual review versus the subset of records reporting possible adverse events. In one example, the manufacturer's risk tolerance 131 (see also Figure SB) may adjust the rule value threshold in the rule metric 124 making it more strict or lax by increasing the value in the rule metric 124 used when executing. In another example, the manufacturer's risk tolerance may adjust the rule value threshold by a percentage based on how much risk is applied across the board, e.g., reducing the bounds of the threshold by fifty percent (50%). The effect of changing these thresholds may expand or reduce the sample size of the data sets and provide a larger or smaller sample size when performing statistical analysis.

The rules and fields analysis engine 130 outputs the statistically significant segment, or subset, of the population data sets 142 as a data structure 132 to the eCRF generation engine 150. The eCRF generation engine 150 generates an eCRF study build, i.e. the process of creating the fields of the eCRF and populating it with values to perform a study, based on the critical fields, patient population data, and base patient data values. The eCRF generation engine 150 takes each column in a row of a data set and enters the value in the corresponding field in the study build. In some illustrative embodiments, this populating of the eCRF can be performed by an SQL import or an API. The eCRF study build is a metadata based template for specifying the critical fields and other information needed to demonstrate compliance with medical device rules 122. The eCRF generation engine 150 populates the eCRF study build with the population segment data, i.e. subset of population data sets 142, needed for medical device as determined based on the rule metric data structure 124 and the critical fields data structure 126, to thereby generate a populated eCRF.

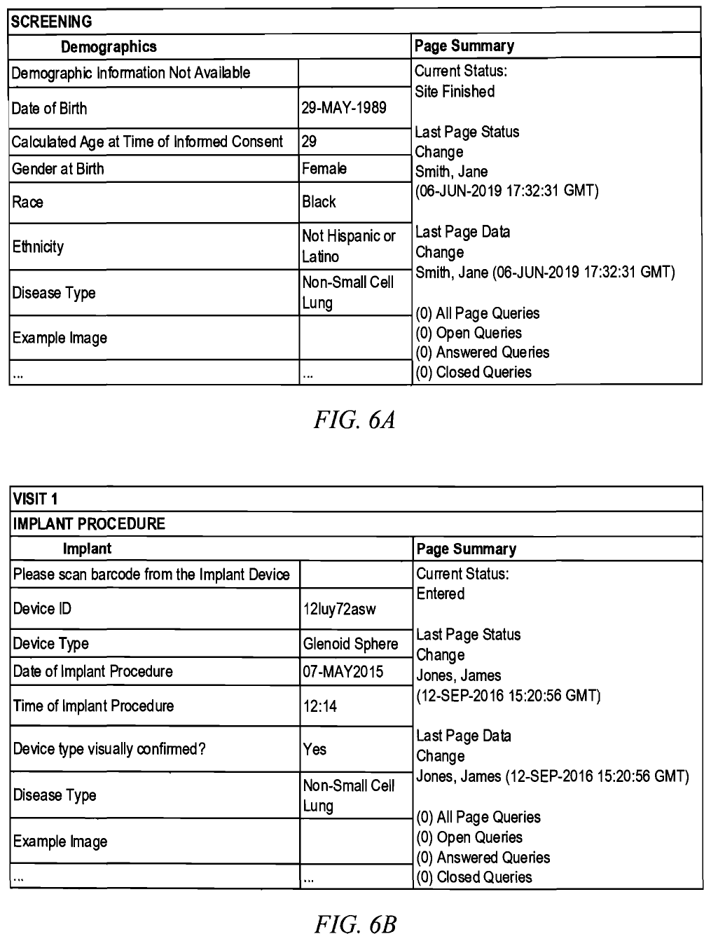

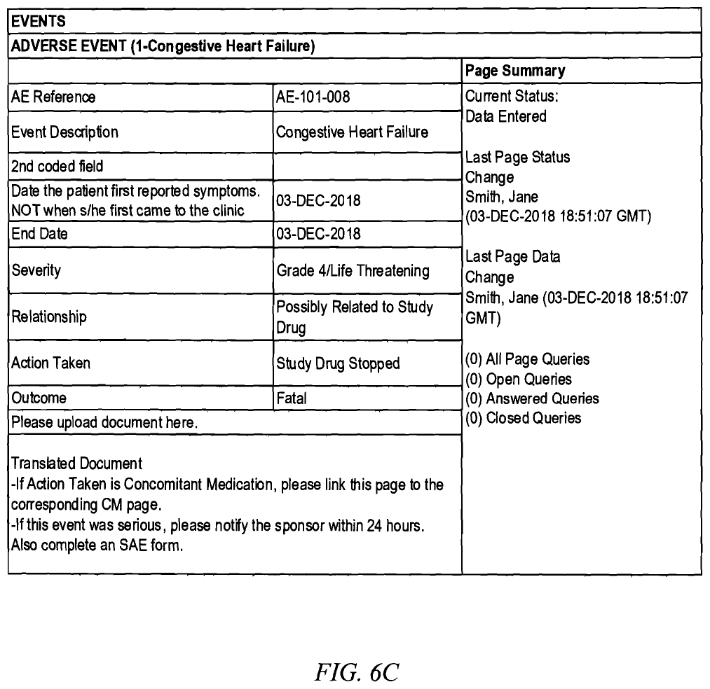

FIGS. 6A-6C are example diagrams of portions of an automatically generated electronic case report form (eCRF) in accordance with one illustrative embodiment. FIG. 6A is an example diagram of a portion of demographics information for an eCRF, where the fields, e.g., date of birth, calculated age at time of informed consent, race, gender, etc., are all standardized fields corresponding to the common schema 112 which are determined to be critical fields 126. The data populating the fields is obtained from patient population data sets 142 corresponding to the subset of population data 134. FIG. 6B is an example diagram of a portion of an eCRF corresponding to a medical device based procedure, in the depicted example an implant procedure since the medical device is an implant. The fields shown in FIG. 6B may be obtained from standardized medical device characteristic fields corresponding to the common schema 112, and which are critical fields 126. Again, the data populating these fields is obtained from the patient population data sets 142 corresponding to the subset of population data 134 where the medical device was indicated to be present in a reported adverse event.

FIG. 6C is an example diagram of a portion of an eCRF corresponding to an adverse event occurrence. The fields shown in FIG. 6C may be obtained from standardized fields corresponding to the common schema 112, and which are critical fields 126. Again, the data populating these fields is obtained from the patient population data sets 142 corresponding to the subset of population data 134 where the medical device was indicated to be present in a reported adverse event.

Once a populated eCRF is generated, it may be transmitted to an appropriate computing system 160 associated with an oversight organization, e.g., the U.S. FDA. The eCRF may be used at the computing system 160 to populate an existing database of eCRF data for various medical devices. The oversight organization computer system 160 may then process and analyze the populated eCRF present in the database to identify any issues with the performance and/or safety of the medical device and may transmit a response back to the automated medical device engine 100 and/or a manufacturer/device provider computing system to inform the manufacturer/device provider of the potential issues, if any, of the medical device. In some illustrative embodiments, the oversight organization computing system 160 may present the populated eCRF to a user via a user interface so that the user may manually review and evaluate the eCRF to determine any issues with the medical device that need to be addressed, and then send communications to appropriate parties to inform them of these issues.

Thus, the illustrative embodiments provide mechanisms for performing automated medical device regulation risk assessment in response to new and/or modified medical device regulations being promulgated by oversight organizations. With regard to this risk assessment, in some illustrative embodiments, there are essentially two risk factors: a first risk factor representing the “riskiness” of the medical device, e.g., a class III medical device represents a greater risk in terms of the medical device's invasiveness in the care of a patient, and a second risk factor representing a risk aversion of the manufacturer of the medical device. Both risks affect the sample size that will be selected for medical review. For example, a class III medical device drives a broader inclusion of attributes that are considered critical for segmenting the population data sets 142 for medical review and thereby, provide a finer grain differentiation of patient records. A class II medical device requires less differentiation and therefore, a smaller sample set needs to be medically reviewed.

Additionally, the manufacturer's risk tolerance, as specified in the risk profile 131, may further drive variability in the sample set selection. As one example, the following risk tolerance parameters may be indicated in a manufacturer's risk tolerance profile for a sample set selection based on medical device class and patient record analysis:
Pacemaker (high-risk): 40% of population to be selected for review
Knee replacement (medium-risk): 30% of population to be selected for review
Catheter (low-risk): 10% of population to be selected for review
   A medical device manufacturer who is risk adverse may want to randomly sample some portion of the remaining population to increase the number of records for all categories, a specific category or even a particular device.

The automated medical device risk assessment mechanisms of the illustrative embodiments determine the most critical fields in patient data and medical device data that are relevant to the medical device rules and simulates patient studies by identifying sub-sets of patient population data sets that are statistically significant to these critical fields and medical device rules. The critical fields and statistically significant segments or sub-sets of the patient population data sets are used to generate an electronic case report form (eCRF) which is then populated with actual data from the sub-sets of patient population data sets to thereby generate a populated eCRF for transmission to an oversight organization computing system for use in ensuring safe and proper performance of medical devices in accordance with the new and/or modified medical device regulations. Thus, the present invention significantly reduces the requirement for medical device manufacturers and providers to perform medical studies of medical devices when medical device rules are created and/or changed by oversight organizations or when the medical device manufacturers and providers seek to deploy their medical devices in new regions or markets where such oversight organizations have jurisdiction.

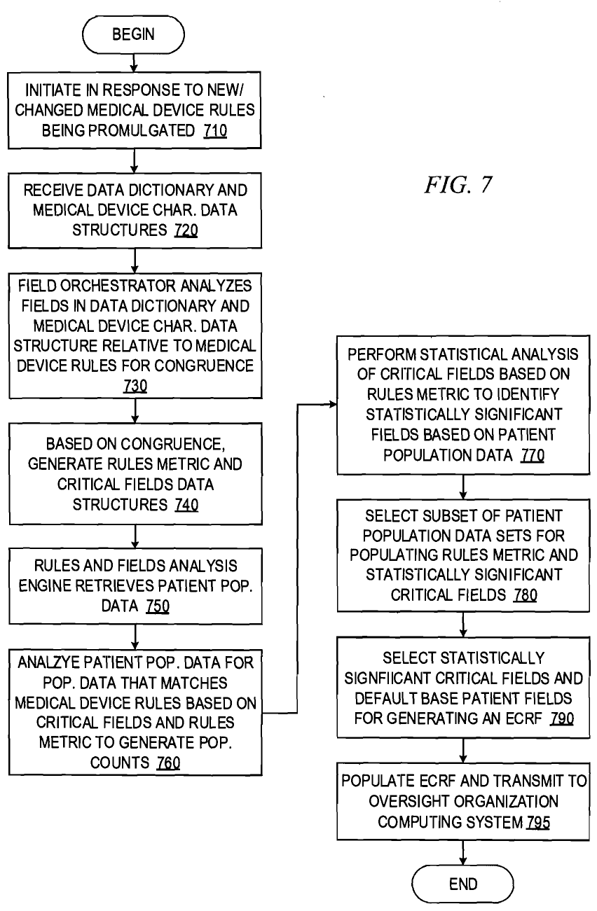

FIG. 7 is a flowchart outlining an example operation of an automated medical device reporting (medical device) engine in accordance with one illustrative embodiment. As shown in FIG. 7, the operation is initiated in response to new changed medical device rules being promulgated by an oversight organization and these medical device rules having been implemented as a set of computer useable medical device rules data structures, such as rules data structure 122 in FIG. 1 (step 710). A data dictionary for patient data and device characteristics data structure for the medical device of interest are received (step 720) and the field orchestrator engine analyzes the fields specified in the data dictionary and device characteristics data structures relative to the medical device rules to determine congruence (step 730). Based on this congruence, the field orchestrator engine generates a rules metric data structure and critical fields data structure which are output to the rules and fields analysis engine (step 740).

The rules and fields analysis engine retrieves patient population data from a patient population data source computing system (step 750) and analyzes this patient population data for patient population data that matches the medical device rules based on the critical fields and rules metric data structures to generate population counts (step 760). The rules and fields analysis engine performs statistical analysis of the critical fields based on the rules metric data structure to identify the statistically significant fields based on the patient population data (step 770). The rules and fields analysis engine then selects a subset of the patient population data sets, i.e. a population segment, for populating information regarding the rules metric and statistically significant critical fields (step 780). Based on the rules metric, and the statistical analysis, statistically significant critical fields and default base patient fields are selected for generating an electronic case report form (eCRF) for medical device (step 790). Thereafter, corresponding patient and medical device data from the selected population segment are used to populate the eCRF and transmit the populated eCRF to an oversight organization computing system for storage in a medical device database and/or review by automated or manual processes at the oversight organization computing system (step 795). The operation then terminates.

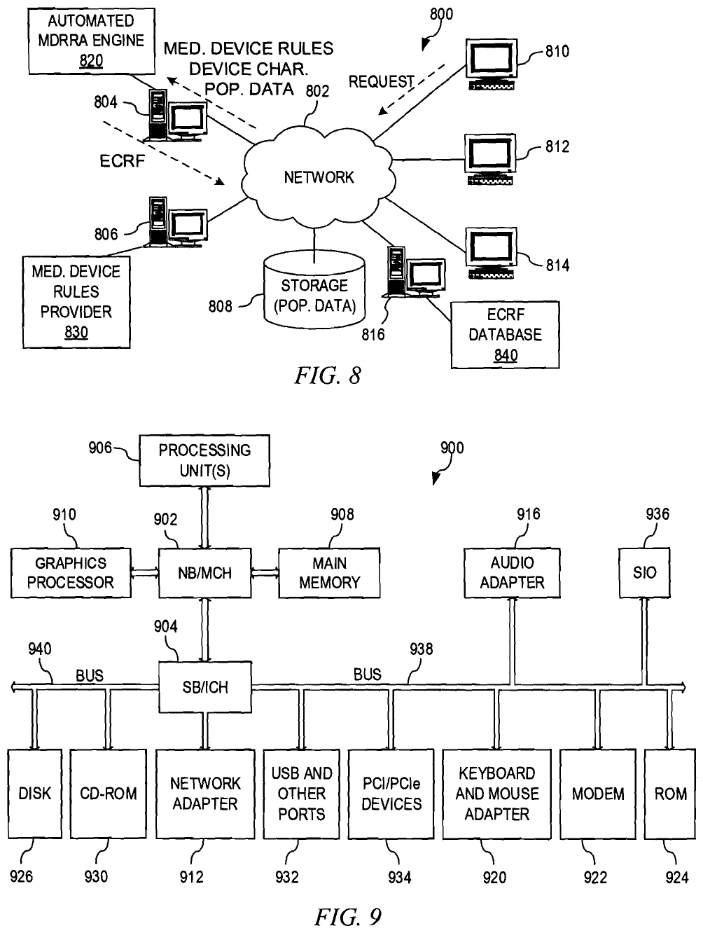

As can be appreciated from the above description and the depictions in the figures, the illustrative embodiments are directed to an improved computing tool for automated medical device reporting to oversight organization computing systems, and thus, may be utilized in many different types of data processing environments. In order to provide a context for the description of the specific elements and functionality of the illustrative embodiments, FIGS. 8 and 9 are provided hereafter as example environments in which aspects of the illustrative embodiments may be implemented. It should be appreciated that FIGS. 8 and 9 are only examples and are not intended to assert or imply any limitation with regard to the environments in which aspects or embodiments of the present invention may be implemented. Many modifications to the depicted environments may be made without departing from the spirit and scope of the present invention.

FIG. 8 depicts a pictorial representation of an example distributed data processing system in which aspects of the illustrative embodiments may be implemented. Distributed data processing system 800 may include a network of computers in which aspects of the illustrative embodiments may be implemented. The distributed data processing system 800 contains at least one network 802, which is the medium used to provide communication links between various devices and computers connected together within distributed data processing system 800. The network 802 may include connections, such as wire, wireless communication links, or fiber optic cables.

In the depicted example, servers 804 and server 806 are connected to network 802 along with storage unit 808. In addition, clients 810, 812, and 814 are also connected to network 802. These clients 810, 812, and 814 may be, for example, personal computers, network computers, or the like. In the depicted example, server 804 provides data, such as boot files, operating system images, and applications to the clients 810, 812, and 814. Clients 810, 812, and 814 are clients to server 804 in the depicted example. Distributed data processing system 800 may include additional servers, clients, and other devices not shown.

In the depicted example, distributed data processing system 800 is the Internet with network 802 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, the distributed data processing system 800 may also be implemented to include a number of different types of networks, such as for example, an intranet, a local area network (LAN), a wide area network (WAN), or the like. As stated above, FIG. 8 is intended as an example, not as an architectural limitation for different embodiments of the present invention, and therefore, the particular elements shown in FIG. 8 should not be considered limiting with regard to the environments in which the illustrative embodiments of the present invention may be implemented.

As shown in FIG. 8, one or more of the computing devices, e.g., server 804, may be specifically configured to implement an automated medical device reporting (medical device) engine 820. The configuring of the computing device may comprise the providing of application specific hardware, firmware, or the like to facilitate the performance of the operations and generation of the outputs described herein with regard to the illustrative embodiments. The configuring of the computing device may also, or alternatively, comprise the providing of software applications stored in one or more storage devices and loaded into memory of a computing device, such as server 804, for causing one or more hardware processors of the computing device to execute the software applications that configure the processors to perform the operations and generate the outputs described herein with regard to the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of the illustrative embodiments and is not a general purpose computing device. Moreover, as described herein, the implementation of the mechanisms of the illustrative embodiments improves the functionality of the computing device and provides a useful and concrete result that facilitates automated medical device reporting that significantly reduces resource costs for medical device manufacturers/providers in complying with new and modified medical device reporting rules issued by oversight organizations by providing mechanisms to satisfy such medical device rules using simulated cases generated from already obtained patient population data after automatically determining the critical fields and statistically significant fields and subset of patient population data that satisfies the medical device rules requirements.

In the context of FIG. 8, it should be appreciated that the automated medical device regulation risk assessment (MDRRA) engine 820, which may operate in the manner previous described above with regard to MDRRA engine 100 in FIG. 1, for example, may be implemented on server 804 and may operate in response to a triggering condition having been detected. The triggering condition may be the issuance of new or updated medical device rules by an oversight organization, a request received to perform automated medical device for a medical device, or the like. For example, a user of a client computing device 810 may send a request to the server 804 specifying the medical device for which automated medical device is to be performed and may provide a medical device characteristics data structure providing a specification of the characteristics of the medical device. In response to receiving the request, the automated MDRRA engine 820 may access currently applicable medical device rules from a medical device rules provider 830, such as may be provided on another server computing system 806 which may be accessible via the network 802. It should be appreciated that an oversight organization may publish new and/or modified medical device rules by transmitting data structures to the medical device rules provider 830, or may themselves by the medical device rules provider 830, to the appropriate computing systems, e.g., server 806, for distribution of the medical device rules to users, such as the automated medical device engine 820 of the illustrative embodiments.

In other illustrative embodiments, the automated MDRRA engine 820 operation may be automatically initiated in response to new or modified medical device rules being published by the oversight organization. That is, in response the oversight organization deploying new/updated medical device rules to a medical device rules provider 830, the new/updated medical device rules may be pushed/pulled to the automated MDRRA engine 820 which may then initiate its automated medical device operations for each registered medical device present a registry of the automated MDRRA engine 820, where this registry may comprise medical device characteristics data structures for the various registered medical devices.

Thus, the automated MDRRA engine 820 receives or otherwise accesses the new/updated medical device rules from the medical device rules provider 830, medical device characteristic data from a medical device manufacturer/provider computing system, such as another server computing system (not shown) accessible via the network 802, and patient population data from the network storage 808 or other computing system (not shown). The automated MDRRA engine 820 then generates, through the processes described previously with regard to FIGS. 1 and 7 above, a populated eCRF that may be output to an oversight organization computing system, such as computing device 816, for storage in an eCRF database 840 for later analysis and evaluation by automated and/or manual processes to ensure proper performance and safe operation of medical devices with subsequent communications being sent to report to medical device manufacturers/providers issues with the medical devices that need to be addressed, if any.

As noted above, the mechanisms of the illustrative embodiments utilize specifically configured computing devices, or data processing systems, to perform the operations for automated medical device reporting. These computing devices, or data processing systems, may comprise various hardware elements which are specifically configured, either through hardware configuration, software configuration, or a combination of hardware and software configuration, to implement one or more of the systems/subsystems described herein. FIG. 9 is a block diagram of just one example data processing system in which aspects of the illustrative embodiments may be implemented. Data processing system 900 is an example of a computer, such as server 804 in FIG. 8, in which computer usable code or instructions implementing the processes and aspects of the illustrative embodiments of the present invention may be located and/or executed so as to achieve the operation, output, and external effects of the illustrative embodiments as described herein.

In the depicted example, data processing system 900 employs a hub architecture including north bridge and memory controller hub (NB/MCH) 902 and south bridge and input/output (I/O) controller hub (SB/ICH) 904. Processing unit 906, main memory 908, and graphics processor 910 are connected to NB/MCH 902. Graphics processor 910 may be connected to NB/MCH 902 through an accelerated graphics port (AGP).

In the depicted example, local area network (LAN) adapter 912 connects to SB/ICH 904. Audio adapter 916, keyboard and mouse adapter 920, modem 922, read only memory (ROM) 924, hard disk drive (HDD) 926, CD-ROM drive 930, universal serial bus (USB) ports and other communication ports 932, and PCI/PCIe devices 934 connect to SB/ICH 904 through bus 938 and bus 940. PCI/PCIe devices may include, for example, Ethernet adapters, add-in cards, and PC cards for notebook computers. PCI uses a card bus controller, while PCIe does not. ROM 924 may be, for example, a flash basic input/output system (BIOS).

HDD 926 and CD-ROM drive 930 connect to SB/ICH 904 through bus 940. HDD 926 and CD-ROM drive 930 may use, for example, an integrated drive electronics (IDE) or serial advanced technology attachment (SATA) interface. Super I/O (SIO) device 936 may be connected to SB/ICH 904.

An operating system runs on processing unit 906. The operating system coordinates and provides control of various components within the data processing system 900 in FIG. 9. As a client, the operating system may be a commercially available operating system such as Microsoft® Windows 10®. An object-oriented programming system, such as the Java™ programming system, may run in conjunction with the operating system and provides calls to the operating system from Java™ programs or applications executing on data processing system 900.

As a server, data processing system 900 may be, for example, an IBM eServer™ System p® computer system, Power™ processor based computer system, or the like, running the Advanced Interactive Executive (AIX®) operating system or the LINUX® operating system. Data processing system 900 may be a symmetric multiprocessor (SMP) system including a plurality of processors in processing unit 906. Alternatively, a single processor system may be employed.

Instructions for the operating system, the object-oriented programming system, and applications or programs are located on storage devices, such as HDD 926, and may be loaded into main memory 908 for execution by processing unit 906. The processes for illustrative embodiments of the present invention may be performed by processing unit 906 using computer usable program code, which may be located in a memory such as, for example, main memory 908, ROM 924, or in one or more peripheral devices 926 and 930, for example.

A bus system, such as bus 938 or bus 940 as shown in FIG. 9, may be comprised of one or more buses. Of course, the bus system may be implemented using any type of communication fabric or architecture that provides for a transfer of data between different components or devices attached to the fabric or architecture. A communication unit, such as modem 922 or network adapter 912 of FIG. 9, may include one or more devices used to transmit and receive data. A memory may be, for example, main memory 908, ROM 924, or a cache such as found in NB/MCH 902 in FIG. 9.

As mentioned above, in some illustrative embodiments the mechanisms of the illustrative embodiments may be implemented as application specific hardware, firmware, or the like, application software stored in a storage device, such as HDD 926 and loaded into memory, such as main memory 908, for executed by one or more hardware processors, such as processing unit 906, or the like. As such, the computing device shown in FIG. 9 becomes specifically configured to implement the mechanisms of the illustrative embodiments and specifically configured to perform the operations and generate the outputs described herein with regard to the automated medical device reporting engine.

Those of ordinary skill in the art will appreciate that the hardware in FIGS. 8 and 9 may vary depending on the implementation. Other internal hardware or peripheral devices, such as flash memory, equivalent non-volatile memory, or optical disk drives and the like, may be used in addition to or in place of the hardware depicted in FIGS. 8 and 9. Also, the processes of the illustrative embodiments may be applied to a multiprocessor data processing system, other than the SMP system mentioned previously, without departing from the spirit and scope of the present invention.

Moreover, the data processing system 900 may take the form of any of a number of different data processing systems including client computing devices, server computing devices, a tablet computer, laptop computer, telephone or other communication device, a personal digital assistant (PDA), or the like. In some illustrative examples, data processing system 900 may be a portable computing device that is configured with flash memory to provide non-volatile memory for storing operating system files and/or user-generated data, for example. Essentially, data processing system 900 may be any known or later developed data processing system without architectural limitation.

Thus, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising a processor and a memory, the memory comprising instructions which are executed by the processor to cause the processor to implement an automated medical device regulation risk analysis (MDRRA) engine, wherein the method comprises:
receiving, by the automated MDRRA engine, compliance rules for a medical device, wherein the compliance rules specify parameters for medical device efficacy;
receiving, by the automated MDRRA engine, at least one data dictionary for a patient data source and medical device characteristics data for the medical device;
automatically determining, by a field orchestration engine of the automated MDRRA engine, based on the compliance rules and the at least one data dictionary, critical fields of patient data and medical device characteristics data for demonstrating medical device efficacy in accordance with the compliance rules;
automatically determining, by a rules and fields analysis engine of the automated MDRRA engine, based on the critical fields, one or more patient population data subsets for a plurality of patients from a patient population data set;
automatically selecting, by the rules and fields analysis engine of the automated MDRRA engine, a population segment from the one or more patient population data sets to populate an electronic case report form (eCRF) study build; and
automatically populating, by an eCRF generation engine of the automated MDRRA engine, fields of the eCRF with data from the population data sets corresponding to the selected population segment.

2. The method of claim 1, wherein receiving the at least one data dictionary further comprises mapping, by the field orchestrator engine of the automated MDRRA engine, a data dictionary for a patient data source, and medical device characteristics of a medical device, to common schema fields of a common schema.

3. The method of claim 2, wherein automatically determining critical fields of patient data and medical device characteristics data for demonstrating medical device efficacy in accordance with the compliance rules further comprises analyzing, by the field orchestrator engine, the compliance rules and the common schema fields for congruence to produce rules metric data and critical fields data, wherein the rules metric data specifies checks against one or more thresholds for identifying normal/abnormal values in patient data, and wherein the critical fields data specifies critical fields in the patient data and medical device characteristics for evaluating the rules metric data.

4. The method of claim 1, wherein automatically selecting a population segment further comprises:
analyzing, by the rules and fields analysis engine of the automated MDRRA engine, the one or more patient population data subsets at least by matching portions of the patient population data set to one or more of the compliance rules to identify adverse event specific cases and population counts; and
determining, by the rules and fields analysis engine, based on the identified adverse event specific cases and population counts, a statistically significant population segment required for specific adverse event cases based on the rules metric data for the critical fields.

5. The method of claim 1, wherein generating the eCRF further comprises:
selecting, by the rules and fields analysis engine, a subset of the critical fields and corresponding patient data fields in the patient data for an original medical device study; and
generating, by the eCRF generation engine, the eCRF based on the subset of critical fields and corresponding patient data fields.

6. The method of claim 1, wherein selecting the population segment comprises determining, for a specific adverse event type, the population segment based on at least one threshold for population segments per compliance rule and critical field.

7. The method of claim 6, wherein selecting a population segment comprises selecting a population segment in which statistically significant field criteria is within a specified manufacturer risk tolerance threshold, based on at least one of sampling size, standard deviation, significance level.

8. The method of claim 1, wherein automatically determining, by a rules and fields analysis engine of the automated MDRRA engine, based on the critical fields, one or more patient population data subsets for a plurality of patients from a patient population data set comprises:
matching one or more of the critical fields to portions of patient population data in the patient population data set; and
performing a statistical analysis of the patient population data, in the patient population data set, to determine one or more patient population data subsets meeting one or more criteria specifying statistically significant subsets.

9. The method of claim 8, wherein automatically selecting, by the rules and fields analysis engine of the automated MDRRA engine, a population segment from the one or more patient population data sets to populate the eCRF study build comprises generating a union of the one or more patient population data subsets.

10. The method of claim 8, wherein the matching is performed for portions of the patient population data set corresponding to a specified injury type.

11. A computer program product comprising a computer readable storage medium having a computer readable program stored therein, wherein the computer readable program, when executed on a computing device, causes the computing device to implement an automated medical device regulation risk analysis (MDRRA) engine, and to:
receive, by the automated MDRRA engine, compliance rules for a medical device, wherein the compliance rules specify parameters for medical device efficacy;
receive, by the automated MDRRA engine, at least one data dictionary for a patient data source and medical device characteristics data for the medical device;
automatically determine, by a field orchestration engine of the automated MDRRA engine, based on the compliance rules and the at least one data dictionary, critical fields of patient data and medical device characteristics data for demonstrating medical device efficacy in accordance with the compliance rules;
automatically determine, by a rules and fields analysis engine of the automated MDRRA engine, based on the critical fields, one or more patient population data subsets for a plurality of patients from a patient population data set;
automatically select, by the rules and fields analysis engine of the automated MDRRA engine, a population segment from the one or more patient population data sets to populate an electronic case report form (eCRF) study build; and
automatically populate, by an eCRF generation engine of the automated MDRRA engine, fields of the eCRF with data from the population data sets corresponding to the selected population segment.

12. The computer program product of claim 11, wherein the computer readable program further causes the computing device to receive the at least one data dictionary further at least by mapping, by the field orchestrator engine of the automated MDRRA engine, a data dictionary for a patient data source, and medical device characteristics of a medical device, to common schema fields of a common schema.

13. The computer program product of claim 12, wherein the computer readable program further causes the computing device to automatically determine critical fields of patient data and medical device characteristics data for demonstrating medical device efficacy in accordance with the compliance rules further at least by analyzing, by the field orchestrator engine, the compliance rules and the common schema fields for congruence to produce rules metric data and critical fields data, wherein the rules metric data specifies checks against one or more thresholds for identifying normal/abnormal values in patient data, and wherein the critical fields data specifies critical fields in the patient data and medical device characteristics for evaluating the rules metric data.

14. The computer program product of claim 11, wherein the computer readable program further causes the computing device to automatically select a population segment further at least by:
analyzing, by the rules and fields analysis engine of the automated MDRRA engine, the one or more patient population data subsets at least by matching portions of the patient population data set to one or more of the compliance rules to identify adverse event specific cases and population counts; and
determining, by the rules and fields analysis engine, based on the identified adverse event specific cases and population counts, a statistically significant population segment required for specific adverse event cases based on the rules metric data for the critical fields.

15. The computer program product of claim 11, wherein the computer readable program further causes the computing device to generate the eCRF at least by:
selecting, by the rules and fields analysis engine, a subset of the critical fields and corresponding patient data fields in the patient data for an original medical device study; and
generating, by the eCRF generation engine, the eCRF based on the subset of critical fields and corresponding patient data fields.

16. The computer program product of claim 11, wherein the computer readable program further causes the computing device to select the population segment at least by determining, for a specific adverse event type, the population segment based on at least one threshold for population segments per compliance rule and critical field.

17. The computer program product of claim 16, wherein the computer readable program further causes the computing device to select a population segment at least by selecting a population segment in which statistically significant field criteria is within a specified manufacturer risk tolerance threshold, based on at least one of sampling size, standard deviation, significance level.

18. The computer program product of claim 11, wherein the computer readable program further causes the computing device to automatically determine, by a rules and fields analysis engine of the automated MDRRA engine, based on the critical fields, one or more patient population data subsets for a plurality of patients from a patient population data set at least by:
matching one or more of the critical fields to portions of patient population data in the patient population data set; and
performing a statistical analysis of the patient population data, in the patient population data set, to determine one or more patient population data subsets meeting one or more criteria specifying statistically significant subsets.

19. The computer program product of claim 18, wherein the computer readable program further causes the computing device to automatically select, by the rules and fields analysis engine of the automated MDRRA engine, a population segment from the one or more patient population data sets to populate the eCRF study build at least by generating a union of the one or more patient population data subsets.

20. An apparatus comprising:
a processor; and
a memory coupled to the processor, wherein the memory comprises instructions which, when executed by the processor, cause the processor to implement an automated medical device regulation risk analysis (MDRRA) engine, and to:
receive, by the automated MDRRA engine, compliance rules for a medical device, wherein the compliance rules specify parameters for medical device efficacy;
receive, by the automated MDRRA engine, at least one data dictionary for a patient data source and medical device characteristics data for the medical device;
automatically determine, by a field orchestration engine of the automated MDRRA engine, based on the compliance rules and the at least one data dictionary, critical fields of patient data and medical device characteristics data for demonstrating medical device efficacy in accordance with the compliance rules;
automatically determine, by a rules and fields analysis engine of the automated MDRRA engine, based on the critical fields, one or more patient population data subsets for a plurality of patients from a patient population data set;
automatically select, by the rules and fields analysis engine of the automated MDRRA engine, a population segment from the one or more patient population data sets to populate an electronic case report form (eCRF) study build; and
automatically populate, by an eCRF generation engine of the automated MDRRA engine, fields of the eCRF with data from the population data sets corresponding to the selected population segment.
